(19) United States
(12) Patent Application Publication
(10) Pub. No.: US 2007/0208256 A1
(43) Pub. Date: Sep. 6, 2007

(54) Multiple Branch Tubular Prosthesis and Methods

(75) Inventor: Brennan Marilla, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/276,512

(22) Filed: Mar. 3, 2006

(51) Int. Cl. A61B 8/14

(52) U.S. Cl. 600/467

(57) ABSTRACT

A tubular prosthesis having an opening formed in a side wall thereof is positioned in a first passageway in a human body with the opening facing and providing fluid flow to a first branch passageway that branches from the first passageway. A portion of the prosthesis that is in the vicinity of the juncture between the first passageway a second branch passageway is located and a second opening is formed in the located portion of the prosthesis so that the second opening faces the second branch passageway. In another embodiment, a tubular prosthesis is endovascularly delivered and positioned in a first passageway in a human body in the vicinity of a second passageway that branches from the first passageway. An imaging device, which is positioned in the prosthesis, locates the juncture between the first passageway and the second passageway after which a piercing member is advanced from the imaging device to a portion of the prosthesis adjacent to the juncture to form an opening in that portion of the prosthesis.

### FIELD OF THE INVENTION

The invention relates to apparatus and methods for endolumenal delivery of prostheses to branch passageways in a human body.

### BACKGROUND OF THE INVENTION

Tubular prostheses such as stents, grafts, and stent-grafts (e.g., stents having an inner and/or outer covering comprising graft material and which may be referred to as covered stents) have been widely used in treating abnormalities in passageways in the human body. In vascular applications, these devices often are used to replace or bypass occluded, diseased or damaged blood vessels such as stenotic or aneurismal vessels. For example, it is well known to use stent-grafts, which comprise biocompatible graft material (e.g., Dacron® or expanded, porous polytetrafluoroethylene (ePTFE)) supported by a framework (e.g., one or more stent or stent-like structures), to treat or isolate aneurysms. The framework provides mechanical support and the graft material or liner provides a blood barrier.

Aneurysms generally involve abnormal widening of a duct or canal such as a blood vessel and generally appear in the form of a sac formed by the abnormal dilation of the duct or vessel wall. The abnormally dilated wall typically is weakened and susceptible to rupture. Aneurysms can occur in blood vessels such as in the abdominal aorta where the aneurysm generally extends below the renal arteries distally to or toward the iliac arteries.

In treating an aneurysm with a stent-graft, the stent-graft typically is placed so that one end of the stent-graft is situated proximally or upstream of the diseased portion of the vessel and the other end of the stent-graft is situated distally or downstream of the diseased portion of the vessel. In this manner, the stent-graft extends through the aneurismal sac and beyond the proximal and distal ends thereof to replace or bypass the dilated wall. The graft material typically forms a blood impervious lumen to facilitate endovascular exclusion of the aneurysm.

Such prostheses can be implanted in an open surgical procedure or with minimally invasive endovascular approach. Minimally invasive endovascular stent-graft delivery generally is preferred over traditional open surgery techniques where the area of diseased vessel is surgically opened, the vessel bypassed and cut, and a stent-graft sutured into position. The endovascular approach, which has been used to delivery stents, grafts and stent-grafts, generally involves cutting through the skin to access a lumen or vasculature. Alternatively, lumenar or vascular access may be achieved percutaneously via successive dilation at a less traumatic entry point. Once access is achieved, the stent-graft can be routed through the vasculature to the target site. For example, a stent-graft delivery catheter loaded with a stent-graft can be percutaneously introduced into the vasculature (e.g., into a femoral artery) and the stent-graft delivered endovascularly to the aneurysm where it is deployed.

When using a balloon expandable stent-graft, balloon catheters generally are used to expand the stent-graft after it is positioned at the target site. When, however, a self-expanding stent-graft is used, the stent-graft generally is radially compressed or folded and placed at the distal end of a sheath or delivery catheter and allowed to expand upon deployment from the sheath or catheter at the target site. More specifically, a delivery catheter having coaxial inner and outer tubes arranged for relative axial movement therebetween can be used and loaded with a compressed self-expanding stent-graft. The stent-graft is positioned within the distal end of the outer tube (sheath) and in front of the inner tube (plunger). Once the catheter is positioned for deployment of the stent-graft at the target site, the plunger is held stationary and the outer tube withdrawn so that the stent-graft is gradually exposed and allowed to expand. An exemplary stent-graft delivery system is described in U.S. Patent Application Publication No. 2004/0093063, which published on May 13, 2004 to Wright et al. and is entitled Controlled Deployment Delivery System, the disclosure of which is hereby incorporated herein in its entirety by reference.

Although the endovascular approach is much less invasive, and usually requires less recovery time and involves less risk of complication as compared to open surgery, there can be concerns with prosthesis alignment in relatively complex applications such as one involving branch vessels. Branch vessel techniques have involved the delivery of a main device (e.g., a graft or stent-graft) and then a secondary device (e.g., a graft or stent-graft) through a fenestration or side opening in the main device and into a branch vessel.

The procedure becomes more complicated when more than one branch vessel is treated. One example is when an aortic abdominal aneurysm is to be treated and its proximal neck is diseased or damaged to the extent that it cannot support a patent connection with a prosthesis. In this case, grafts or stent-grafts have been provided with fenestrations or openings formed in their side wall below a proximal portion thereof. The fenestrations or openings are aligned with the renal arteries and the proximal portion is secured to the aortic wall above the renal arteries.

To ensure alignment of the prostheses fenestrations and branch vessels, current techniques involve placing guidewires through each fenestration and branch vessel (e.g., artery) prior to releasing the main device or prosthesis. This involves manipulation of multiple wires in the aorta at the same time, while the delivery system and stent-graft are still in the aorta. In addition, an angiographic catheter, which may have been used to provide detection of the branch vessels and preliminary prosthesis positioning, may still be in the aorta. Not only is there risk of entanglement of these components, the preformed prosthesis fenestrations may not properly align with the branch vessels due to differences in anatomy from one patient to another. Custom prostheses having preformed fenestrations or openings based on a patient's CAT scans also are not free from risk. A custom design prosthesis is still subject to a surgeon's interpretation of the scan and may not result in the desired anatomical fit. Further, relatively stiff catheters are used to deliver grafts and stent-grafts and these catheters can reshape the vessel (e.g., artery) in which they are introduced. When the vessel is reshaped, even a custom designed prosthesis may not properly align with the branch vessels.

U.S. Pat. No. 5,617,878 to Taheri discloses a method comprising interposition of a graft at or around the intersection of major arteries and thereafter, use of intravenous ultrasound or angiogram to visualize and measure the point on the graft where the arterial intersection occurs. A laser or cautery device is then interposed within the graft and used to create an opening in the graft wall at the point of the intersection. A stent is then interposed within the graft and through the created opening of the intersecting artery.

There remains a need to develop and/or improve branch vessel apparatus and methods for endolumenal or endovascular applications.

### SUMMARY OF THE INVENTION

The present invention involves improvements in tubular prosthesis delivery and overcomes disadvantages in prior art.

According to one embodiment of the invention, a method of placing a tubular prosthesis in a passageway in a human body comprises positioning a tubular prosthesis having an opening formed in a side wall thereof in a first passageway in a human body with the opening facing and providing fluid flow to a first branch passageway that branches from the first passageway; locating a portion of the prosthesis that is in the vicinity of the juncture between the first passageway and a second branch passageway that branches from the first passageway; and forming in vivo a second opening in the located portion of the prosthesis so that the second opening faces the second branch passageway.

According to another embodiment of the invention, a method of forming an opening in a tubular prosthesis in vivo comprises endovascularly positioning a tubular prosthesis in a first passageway in a human body in the vicinity of a second passageway that branches from the first passageway; positioning an imaging device in the prosthesis and locating the juncture between the first passageway and the second passageway therewith; extending a piercing member from the imaging device to a portion of the prosthesis adjacent to the juncture; and forming an opening in the portion with the piercing member. The method may further include advancing a guidewire from the piercing member into the second passageway.

According to another embodiment of the invention, a method of placing a tubular prosthesis in a passageway in a human body in vivo comprises endovascularly positioning a tubular prosthesis, which has a preformed opening formed in a side wall thereof, in a first passageway in a human body in the vicinity of second and third passageways that branch from the first passageway so that the preformed opening faces and provides fluid flow to the second passageway; positioning an imaging device in the prosthesis and locating the juncture between the first passageway and the third passageway therewith; and extending a piercing member from the imaging device to a portion of the prosthesis adjacent to the juncture; forming an opening in the portion with the piercing member. The method my further include advancing a guidewire from the piercing member into the third passageway.

According to another embodiment of the invention, a system for placing a prosthesis in the vicinity of a branch vessel comprises a tubular prosthesis adapted to be endovascularly delivered through a vessel in a human body; and an imaging catheter adapted to be positioned in the tubular prosthesis and detect a branch vessel that branches from the vessel in which it is placed, the imaging catheter having a piercing member that is extendable therefrom and adapted to form an opening in the tubular prosthesis in the vicinity of the detected branch vessel. The system may further include a guidewire that is extendable from the piercing member.

The above is a brief description of some deficiencies in the prior art and advantages of the present invention. Other features, advantages, and embodiments of the invention will be apparent to those skilled in the art from the following description and accompanying drawings, wherein, for purposes of illustration only, specific forms of the invention are set forth in detail.

### BRIEF DESCRIPTION OF THE DRAWINGS

FIG. 1 diagrammatically illustrates detecting a branch vessel opening from a prosthesis lumen with an imaging catheter having a guidewire delivery system positioned therein in accordance with one embodiment of the invention.

FIG. 2 diagrammatically illustrates piercing the endolumenal prosthesis of FIG. 1 in the vicinity of the detected branch vessel opening.

FIG. 3 diagrammatically illustrates advancing a guidewire from the imaging catheter through the opening formed in the endolumenal prosthesis and shown in FIG. 2.

FIG. 4 diagrammatically illustrates withdrawal of the imaging catheter of FIG. 1 with the guidewire being left in place.

FIG. 5 diagrammatically illustrates a balloon catheter tracked over the guidewire of FIGS. 3 and 4 with its balloon extending through the opening formed in the endolumenal prosthesis.

FIG. 6 diagrammatically illustrates inflating the balloon of FIG. 5 to enlarge the opening formed in the endolumenal prosthesis.

FIG. 7 diagrammatically illustrates withdrawing the balloon catheter after the balloon has been deflated.

FIG. 8 diagrammatically illustrates deploying a branch vessel prosthesis from a catheter tracked over the guidewire, which was deployed as shown in FIGS. 3 and 4.

FIG. 9 depicts the prostheses of FIG. 8 and another branch vessel prosthesis deployed from the preformed opening in the prosthesis of FIG. 1.

FIG. 10 diagrammatically illustrates an imaging catheter system according to one embodiment of the invention.

FIG. 10A is an enlarged view of the distal end portion of the imaging system of FIG. 10 with a piercing element advanced therefrom.

FIG. 10B illustrates the distal end portion of FIG. 10A with a guidewire advanced from the piercing element.

FIG. 11 is a front view of one embodiment of an endolumenal prosthesis suitable for use with the imaging catheter of FIG. 1.

### DETAILED DESCRIPTION

The following description will be made with reference to the drawings where when referring to the various figures, it should be understood that like numerals or characters indicate like elements.

The invention generally involves apparatus and methods for providing access to a branch passageway in a human body. One method involves forming in vivo an opening in the wall of a prosthesis (e.g., an arterial graft or stent-graft), which has been deployed at target location in a passageway in the human body. The prosthesis can be deployed, for example, to treat an aneurysm or an occluded area where branch vessel access is desired. Such access may be required in or around the intersection of a vessel (e.g., the aorta) and other attendant vessels (e.g., major arteries such as the renal, brachiocephalic or carotid arteries).

In one embodiment, an imaging catheter having a device to form an opening in the side wall of a main prosthesis, which has been deployed in a passageway in the human body, is positioned in the prosthesis. One suitable imaging catheter is the PIONEER catheter which is an intravenous ultrasound device (IVUS) manufactured by Medtronic Vascular, Inc. (Santa Rosa, Calif.). The imaging catheter detects an area of the prosthesis that is adjacent to a branch passageway (e.g., a renal artery), which branches from the main passageway in which the prosthesis has been deployed. The imaging catheter opening forming device is manipulated or advanced to form an opening in that area of the prosthesis to provide access to the branch passageway. The imaging catheter also can include a guidewire that can be advanced through the opening. In this case, the guidewire is advanced into the branch passageway and is left in position, while the imaging catheter is withdrawn and removed. A secondary prosthesis delivery catheter then can be passed over the guidewire to deliver a secondary or branch prosthesis in the branch passageway at the juncture of the passageway in which the main prosthesis was deployed and the branch passageway. The secondary prosthesis then can be deployed or positioned to seal the opening formed in the main prosthesis to the branch vessel.

A conventional cutting balloon catheter can be used to widen the opening before introducing the secondary prosthesis into the branch passageway. In this case, the balloon catheter is passed over the guidewire and positioned with the balloon extending into the opening. The balloon is expanded to widen the opening and then deflated and withdrawn. The secondary prosthesis delivery catheter is then passed over the guidewire as described above. Alternatively, a known laser cautery device having at one end a cutting appendage can be used instead of the cutting balloon to form the prosthesis side opening.

In another embodiment, a prosthesis such as a stent-graft having one preformed side opening is placed with that opening opposing an upper side branch passageway or vessel. The imaging catheter (e.g., PIONEER imaging catheter is then used to image and locate a lower branch artery that the prosthesis is blocking. The piercing member is advanced from the imaging catheter to pierce the prosthesis side wall in the vicinity of the lower branch passageway or vessel. A guidewire can then be advanced from the imaging catheter or piercing member and introduced into the lower branch passageway or vessel. The prosthesis side opening or hole can be expanded and a secondary or branch prosthesis deployed as described above to seal the opening to its respective branch passageway or vessel (i.e., the lower branch passageway or vessel). Another secondary prosthesis then is delivered and deployed at the upper branch vessel to seal the preformed prosthesis opening and upper branch vessel.

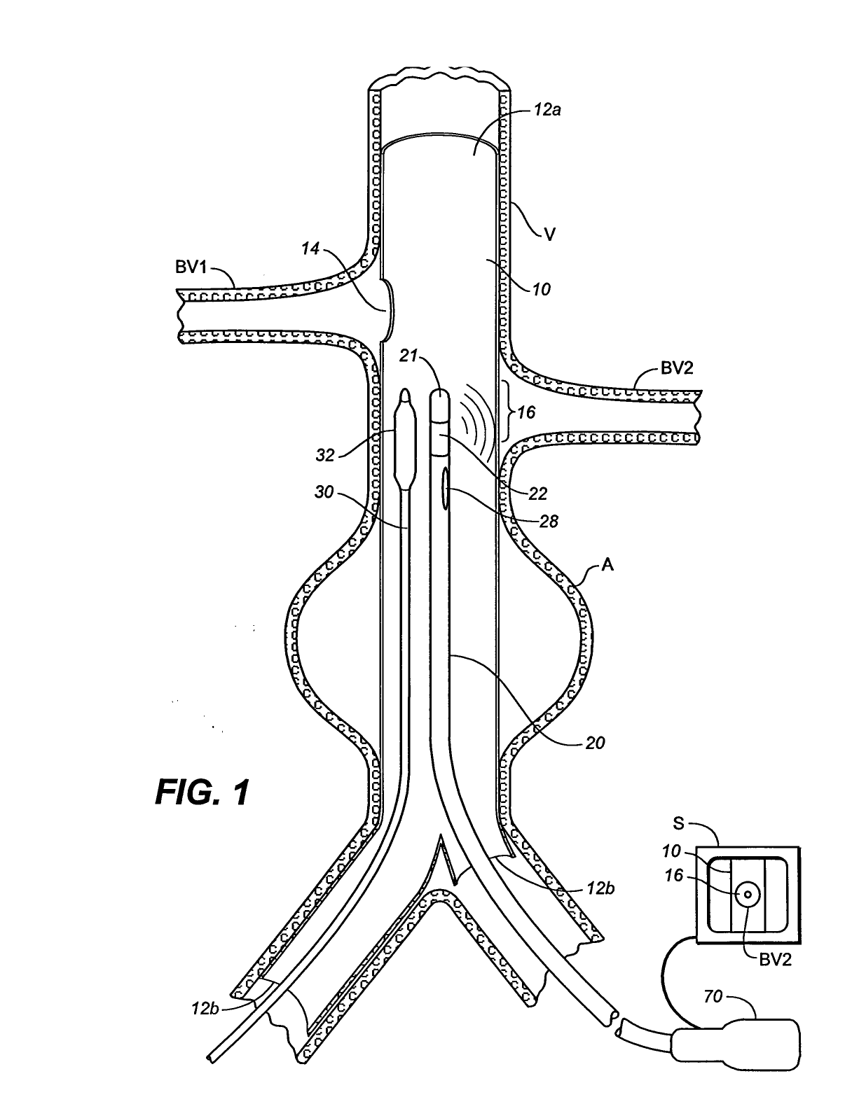

Referring to FIGS. 1-9, one example is shown to illustrate a method according to the invention. In this example, the proximal portion of the prosthesis is secured to the portion of a vessel (e.g., the aorta) proximal to the branch vessels (e.g., renal arteries) due to insufficient aortic proximal neck between the aneurysm and the branch vessels upstream therefrom.

Referring to FIG. 1, a tubular bifurcated prosthesis 10, which can be an expandable or self-expanding bifurcated graft or stent-graft, comprises a tubular wall having proximal and distal end openings 12a and 12b and only one preformed side opening 14. The proximal end of the prosthesis can be scalloped or provided with a cutout as can any of the prosthesis described herein when suitable for the intended application. For example, when the prosthesis is used to bypass an abdominal aortic aneurysm and its proximal portion placed above the renal arteries, a cutout can be provided to allow blood flow to the superior mesentery artery.

Using conventional endovascular graft or stent-graft delivery techniques, the prosthesis is positioned in vessel V with the preformed side opening 14 arranged so that it faces and provides blood flow to upper branch vessel BV1. When the branch vessel is a renal artery, for example, opening 14 provides blood flow to the kidney that the renal artery feeds. Although not shown, prosthesis 10 can have anchoring mechanisms such as collars having tines extending therefrom at the proximal and distal ends thereof to secure the prosthesis to vessel V.

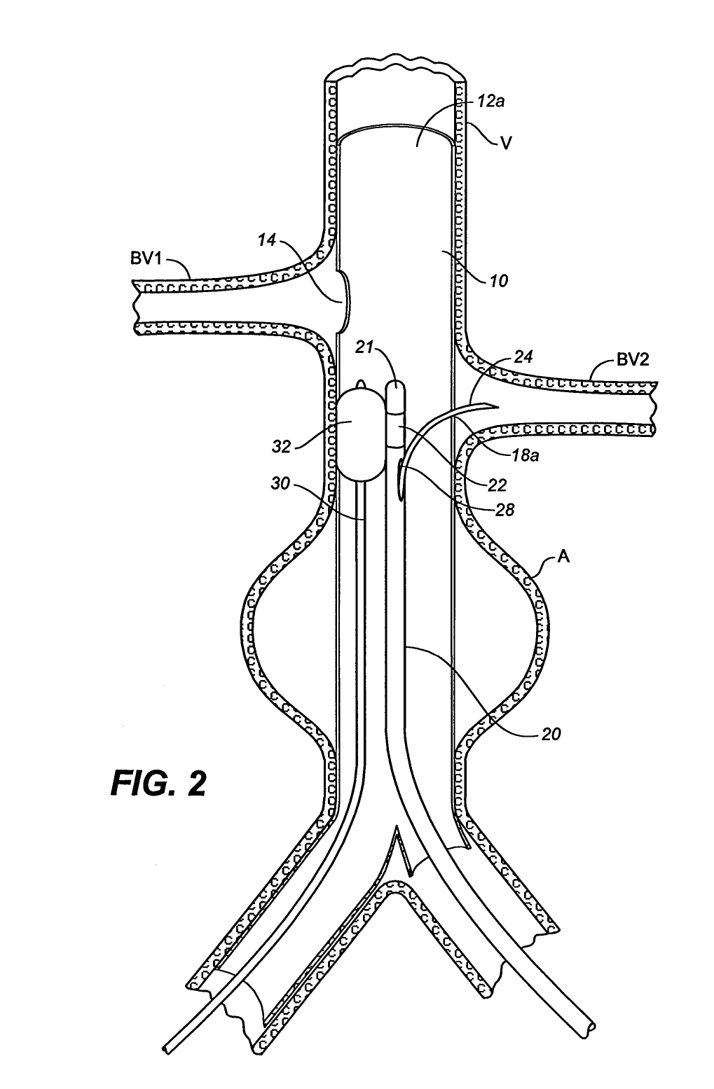

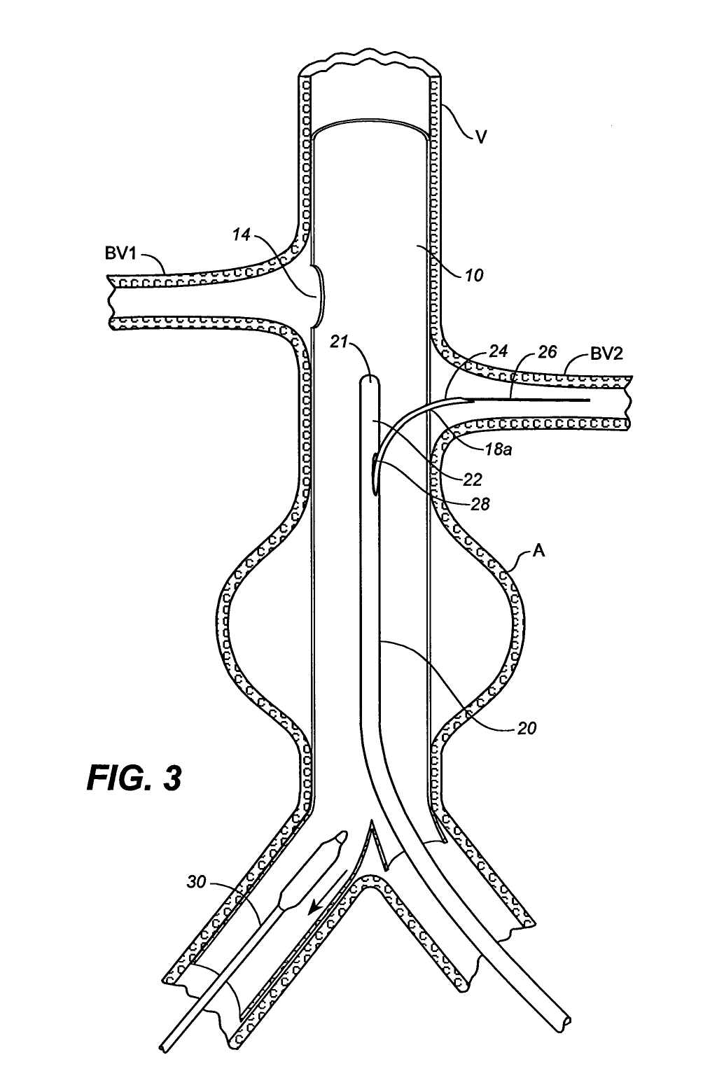

Once prosthesis 10 is in place, imaging catheter 20 is routed endovascularly and positioned inside the prosthesis in the vicinity of the juncture of vessel V and branch vessel BV2 as shown in FIG. 1. It can be introduced through one of the femoral arteries and routed to the desired site (e.g., an area where branch vessel BV2 branches from the vessel V, which may be the aorta). In the illustrative embodiment, imaging catheter 20 is an intravenous ultrasound device (IVUS). Imaging catheter 20 includes a distal end 21, ultrasound head portion or transducer 22, a piercing and guidewire delivery system comprising an extendable piercing member 24 (FIG. 2) and guidewire 26 (FIG. 3) both of which can be advanced through aperture 28 in catheter 20. A balloon catheter 30 having an inflatable balloon 32 can be delivered to the target site using conventional techniques. The balloon catheter can be positioned so that when it is expanded or inflated, it urges imaging catheter toward branch vessel BV2 when piercing member 24 is forced against prosthesis 10 as shown in FIG. 2 and as will be described in further detail below.

Ultrasound emitting and receiving head portion or transducer 22 can transmit signals to an appropriate receptor which processes the signals for imaging on monitor or screen S as is known in the art. Screen S can be placed in the operating area so that the operating surgeon may visualize and measure through ultrasound images the area on prosthesis 10 where the intersection of vessel V and branch vessel BV2 (e.g., the aorta and renal arteries) occurs. More specifically, the emitted and received ultrasound signals are processed so that the surgeon can visualize an area on prosthesis 10, for example area or portion 16, that is adjacent to or at the intersection of vessel V and branch vessel BV2 (e.g., the aorta and renal arteries). The surgeon can measure or mark area 16 as visualized on the monitor or screen using well-known techniques. The surgeon also may visualize the extent and/or nature of any disease or occlusion in the area.

Referring to FIG. 2, the surgeon then advances hollow piercing member 24, which can have a beveled tip as shown, from imaging catheter 20 in a manner such that it extends toward prosthesis wall area or portion 16. The position of the distal end of the imaging catheter can be adjusted if necessary so that when piercing member 24 is further advanced, it penetrates prosthesis 10 in visualized area 16. An opening 18a is created through the prosthesis in the vicinity of the intersection between vessel V and branch vessel BV2. In grafts and stent-grafts the opening or hole is formed through the in the graft material. Since imaging catheter is generally flexible so that it can pass through tortuous vasculature, balloon 32 can be expanded or inflated to provide support for the distal portion of imaging catheter 20 as piercing member 24 is passed through the wall of prosthesis 10 and into branch vessel BV2. Balloon 32 also can inflated or expanded to displace the distal portion of imaging catheter 20 toward area or portion 16 so as to assist in forcing piercing member 24 through the prosthesis. As shown, the distal portion of piercing member 24, which can comprise a hollow needle with a beveled end, has an arc or curved shape when it has been extended. Piercing member 24 can be made from shape memory material and provided with such a preshaped memory set configuration as is known in the art. For example, an end portion of member 24 can be placed in the desired shape (e.g., that shown in FIG. 2) and heated for about 5-15 minutes in a hot salt bath or sand having a temperature of about 480-515° C. It can then be air cooled or placed in an oil bath or water quenched depending on the desired properties. Piercing member 24 can be made from other materials as well such as medical grade stainless steel.

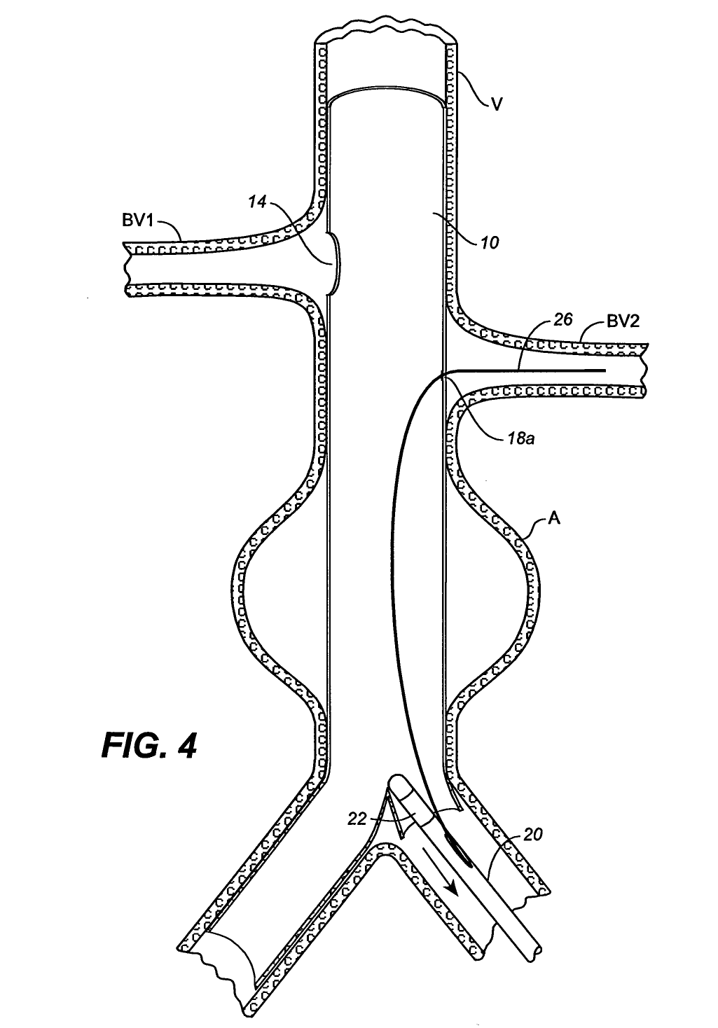

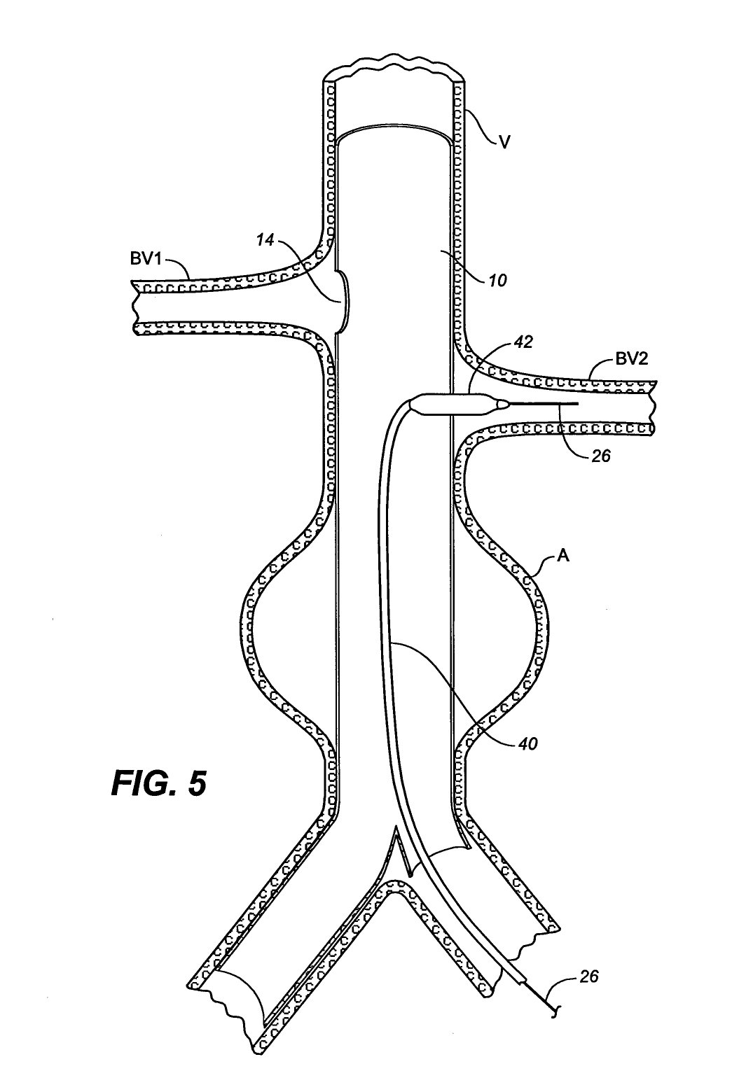

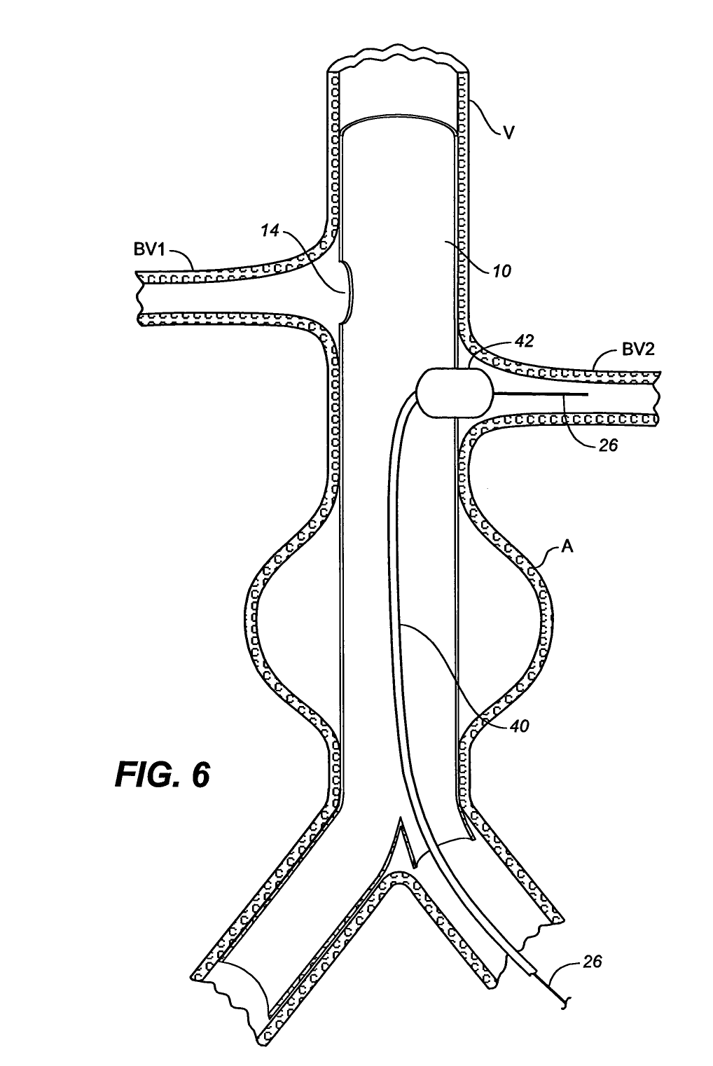

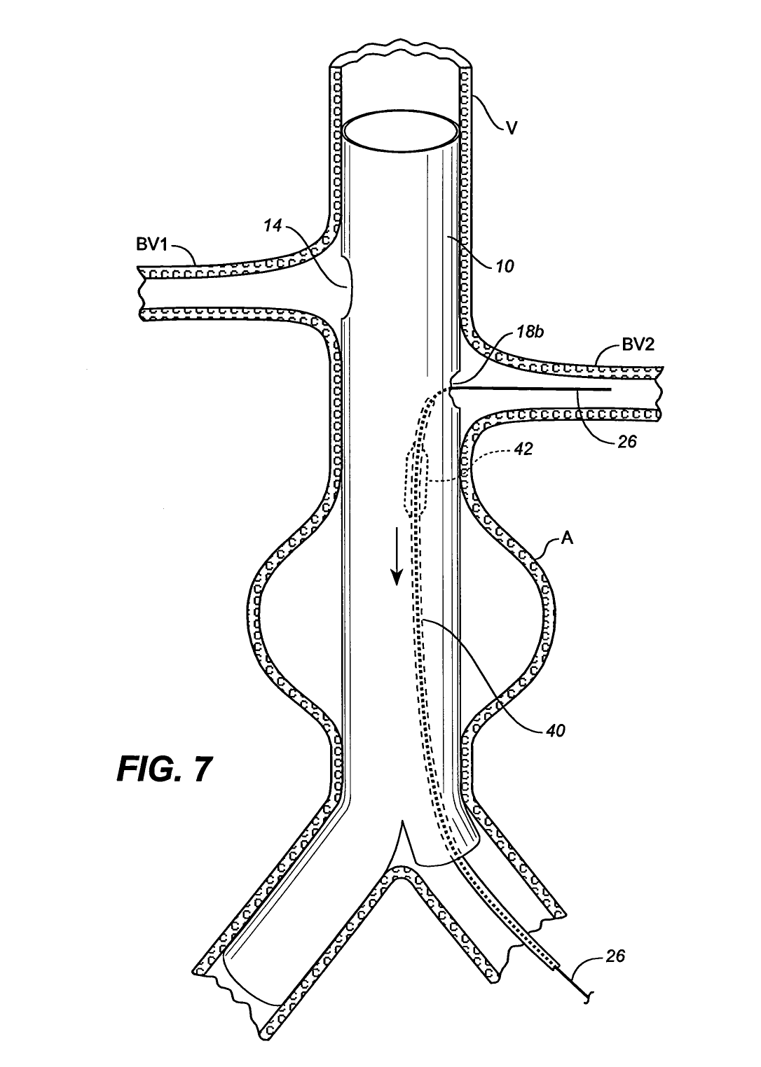

Referring to FIG. 3, guidewire 26 is advanced through piercing member 24 and into branch vessel BV2. Balloon catheter 30 can be deflated and withdrawn before or after the guidewire is advanced. Imaging catheter 20 is then withdrawn, while leaving guidewire 26 in position (FIG. 4) and a balloon catheter such as balloon catheter 40 is passed over guidewire 26 and positioned so that balloon 42, which can be a cutting balloon, extends through opening 18a (FIG. 5). Balloon 42 is then expanded or inflated to enlarge the side opening in prosthesis 10 (FIG. 6) and then deflated and removed leaving enlarged opening 18b (FIG. 7).

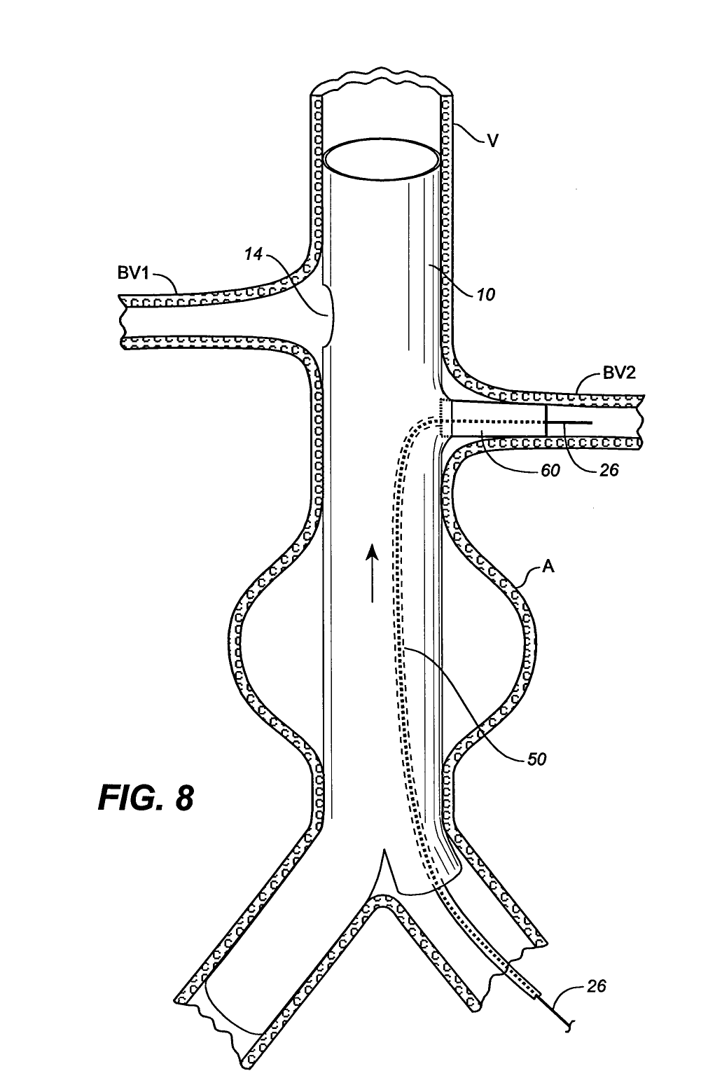

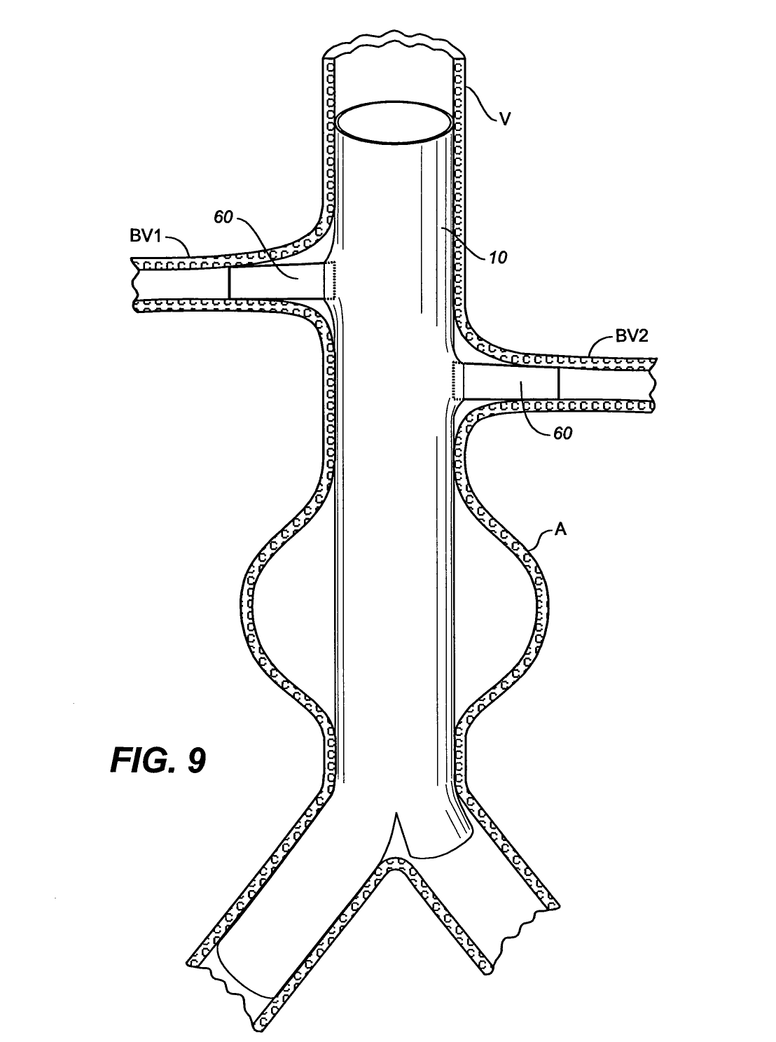

Once opening 18a is completed, the surgeon is in a position to strengthen the opening with a branch prosthesis 60, which is diagrammatically shown in FIG. 8 (such branch stent grafts are known in the art, e.g., WO2005/046526, incorporated herein by reference). Prosthesis delivery catheter 50 is loaded with prosthesis 60, which in this example is a self-expandable stent-graft, and passed over guidewire 26 to position and deploy prosthesis 60 in opening 18b and in branch vessel BV2 to seal the opening and branch vessel. In the case where branch vessel BV2 is a renal artery, the procedure opens the prosthesis side wall to allow blood flow to flow through the renal artery to a respective kidney. In such a case, the rifle like firing of the piercing member and guide wire from the imaging catheter through the prosthesis and into the branch vessel can significantly reduce procedure time. This is especially important when considering that a kidney may not survive more than about 20-30 minutes without blood flow thereto. Another prosthesis 60 is then delivered and deployed in opening 14 and branch vessel BV1 with conventional endolumenal techniques to seal opening 14 and branch vessel as shown in FIG. 9.

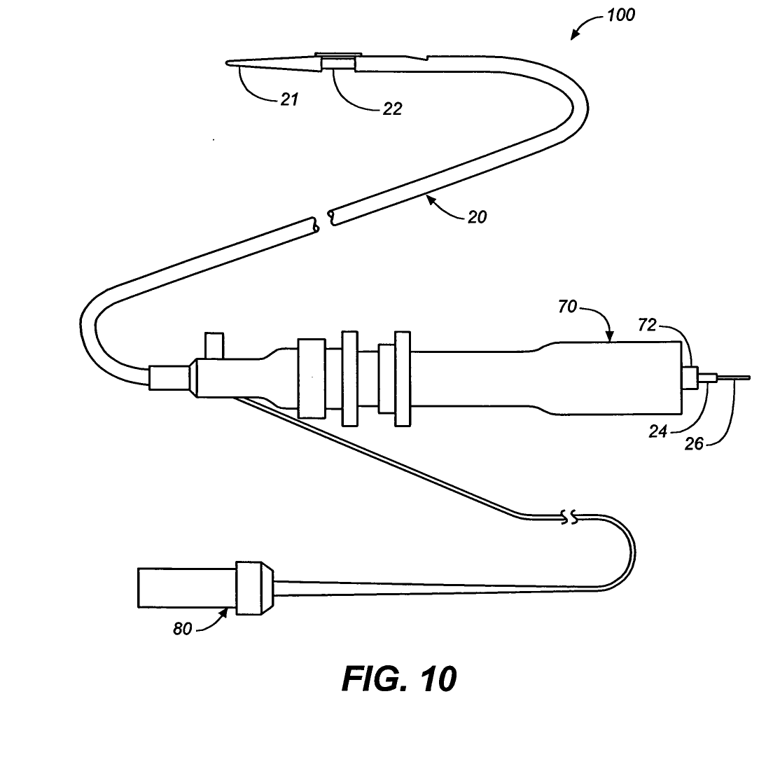

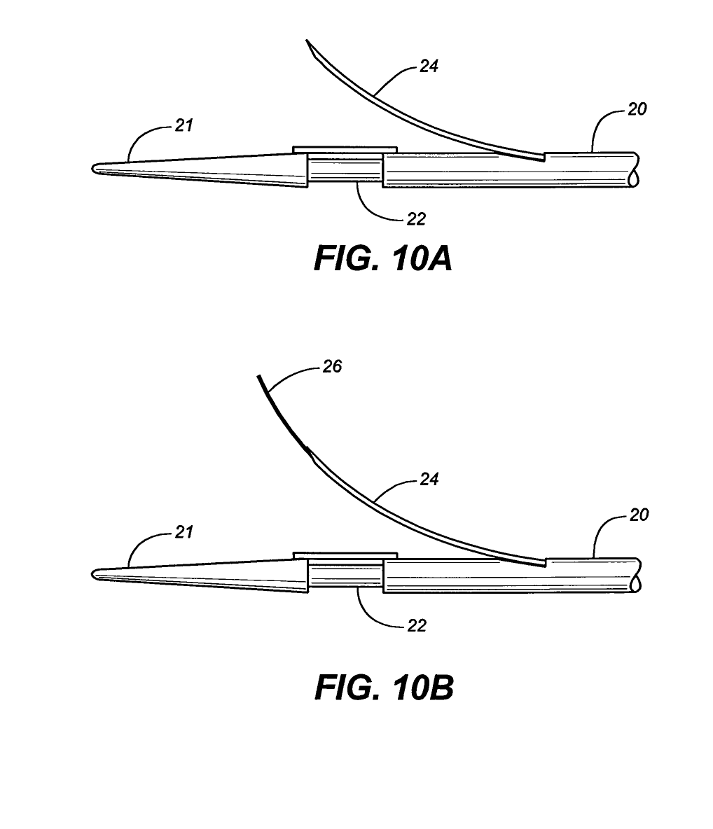

Referring to FIGS. 10, 10a and 10b an imaging catheter system according to the one embodiment of the invention is shown and generally designated with reference numeral 100. System 100 comprises imaging catheter 20, control handle 70 and connector 80 that is adapted to be coupled to an IVUS viewing device in the operating room. Catheter 20 includes tapered tip 21, intravenous ultrasound transducer 22, extendable piercing member 24 and guidewire 26, which can be advanced from aperture 28 (see e.g., FIG. 3). Catheter 20 also comprises a monorail lumen which is relatively short lumen near the end of the catheter that can be threaded over an already placed guide wire, similar to so called Rx delivery catheters used for other devices. Handle 70 includes an inlet 72 for introducing piercing member 24 and guidewire 26 therethrough and to aperture 28. The Medtronic Pioneer catheter provides this functionality.

FIG. 10A shows piercing member 24 advanced therefrom and FIG. 10B shows guidewire 26 advanced from the piercing member 24.

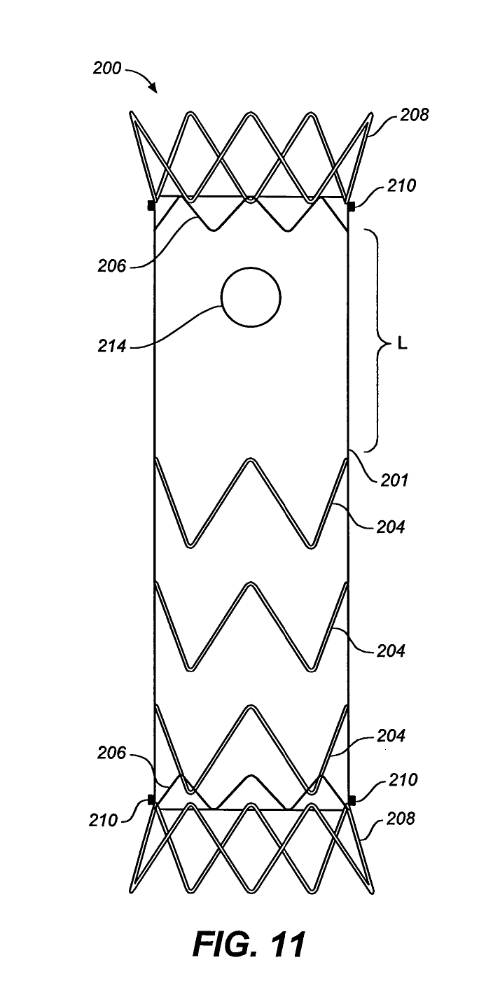

FIG. 11 illustrates an alternate prosthesis configuration in the form of a nonbranching stent-graft. Prosthesis 200 has a tubular portion 201 comprising any suitable graft material, annular undulating wire spring elements or stents 204 which structurally support tubular graft 201 and are secured thereto using conventional techniques. Tubular graft portion 201 can be positioned on the interior and/or exterior of wire spring elements 204. Undulating wire support springs 206 can be provided at either or both ends of tubular graft 201 to provide radial strength and also can be positioned on the interior and/or exterior thereof. Bare springs 208, which are secured to the proximal and distal ends of tubular graft 201, also have an undulating configuration and have a radially outward biased configuration when a free state (e.g., a released state). In this manner, they serve to secure the graft against the wall forming the lumen in which the prosthesis is to be placed. Although springs 208 are shown at both ends of tubular graft 201, only one can be attached to either end of the tubular graft depending on the desired anchoring at the target site. It should be understood, however, that other anchoring means can be used in lieu of springs 208 or in combination with either or both springs 208. The spring elements, support springs and bare springs can be of any suitable material as would be apparent to one of ordinary skill in the art. One suitable material is nitinol. The graft material also can be any suitable material such as Dacron® or expanded, porous polytetrafluoroethylene (ePTFE). Such materials resist tear propagation when piercing member 24 is passed therethrough.

Tubular member 201 includes one preformed opening or fenestration 214, which can be the same as opening or fenestration 14 shown in FIG. 1. Radiopaque ring or ring segment(s) 210 also can be provided to facilitate positioning the stent-graft so that opening or fenestration 214 faces one of a plurality of branch vessel openings. Such markers can be secured to the prosthesis with any suitable biocompatible adhesive.

The proximal portion of the stent-graft can be provided with a stent free zone to facilitate forming in vivo an opening therein. The length L of the stent free zone varies depending on the application and generally is about 20 to 40 mm. For example, when configured for cooperating with the renal arteries, L will be about 30 mm. Alternatively, stents having larger cells or areas between wire curves/apices can be placed in this region to accommodate passing a secondary stent or stent-graft therethrough.

Any feature described in any one embodiment described herein can be combined with any other feature of any of the other embodiments.

Variations and modifications of the devices and methods disclosed herein will be readily apparent to persons skilled in the art. As such, it should be understood that the foregoing detailed description and the accompanying illustrations, are made for purposes of clarity and understanding, and are not intended to limit the scope of the invention, which is defined by the claims appended hereto.

What is claimed is:

1. A method of forming an opening in a tubular prosthesis in vivo comprising:
endovascularly positioning a tubular prosthesis in a first passageway in a human body in the vicinity of a second passageway that branches from the first passageway;
positioning an imaging device in the prosthesis and locating the juncture between the first passageway and the second passageway therewith;
extending a piercing member from the imaging device to a portion of the prosthesis adjacent to the juncture; and
forming an opening in the portion with the piercing member into the second passageway.

2. The method of claim 1 including advancing a guidewire from the piercing member into the second passageway.

3. The method of claim 2 including enlarging the opening.

4. The method of claim 3 wherein a balloon catheter is advanced over the guidewire and into the opening and expanded to enlarge the opening.

5. The method of claim 4 wherein the balloon catheter is withdrawn and a secondary tubular prosthesis advanced over the guidewire and positioned in the opening and second passageway.

6. The method of claim 3 wherein a secondary tubular prosthesis is advanced over the guidewire and positioned in the opening and second passageway.

7. The method of claim 6 wherein the guidewire is withdrawn.

8. The method of claim wherein the tubular prosthesis has a preformed opening formed in a side wall thereof and the tubular prosthesis is positioned in a first passageway in a human body with the opening facing and providing fluid flow to a first branch passageway that branches form the first passageway wherein a secondary tubular prosthesis is delivered over the guide device and positioned in the first passageway with the preformed opening facing and providing fluid flow to a third passageway that branches from the first passageway.

9. The method of claim 8 wherein the first passageway is an artery.

10. The method of claim 9 wherein the first passageway is the aorta.

11. The method of claim 10 wherein the second and third passageways are renal arteries.

12. The method of claim 11 wherein the tubular prosthesis comprises a graft.

13. The method of claim 11 wherein the tubular prosthesis comprises a stent-graft.

14. The method of claim 9 wherein the tubular prosthesis comprises a graft.

15. The method of claim 9 wherein the tubular prosthesis comprises a stent-graft.

16. The method of claim 1 wherein the tubular prosthesis comprises a graft.

17. The method of claim 1 wherein the tubular prosthesis comprises a stent-graft.

18. A method of placing tubular prosthesis in a passageway in a human body comprising:
endovascularly positioning a tubular prosthesis, which has a preformed opening formed in a side wall thereof, in a first passageway in a human body in the vicinity of second and third passageways that branch from the first passageway so that the preformed opening faces and provides fluid flow to the second passageway;
positioning an imaging device in the prosthesis and locating the juncture between the first passageway and the third passageway therewith;
extending a piercing member from the imaging device to a portion of the prosthesis adjacent to the juncture; and
forming an opening in the portion with the piercing member.

19. The method of claim 18 wherein an expandable balloon is placed near the imaging device and expanded to force the piercing member through the prosthesis portion.

20. The method of claim 18 further including advancing a guidewire from the piercing member into the third passageway and withdrawing the imaging device.

21. The method of claim 20 wherein a balloon catheter having a balloon is advanced over the guidewire and the balloon into the portion opening and expanded to enlarge that opening.

22. The method of claim 21 wherein the balloon catheter is deflated and withdrawn and a secondary tubular prosthesis advanced over the guidewire and positioned in the opening and third passageway.

23. The method of claim 27 wherein another secondary tubular prosthesis is positioned in the preformed opening and second passageway.

24. The method of claim 18 wherein the first passageway is an artery.

25. The method of claim 18 wherein the first passageway is the aorta.

26. The method of claim 25 wherein the second and third passageways are renal arteries.

27. The method of claim 18 wherein the tubular prosthesis comprises a graft.

28. The method of claim 18 wherein the tubular prosthesis comprises a stent-graft.

29. A system for placing a prosthesis in the vicinity of a branch vessel comprising:
a tubular prosthesis adapted to be endovascularly delivered through a vessel in a human body; and
an imaging catheter adapted to be positioned in the tubular prosthesis and detect a branch vessel that branches from said vessel in which it is placed, said imaging catheter having a piercing member that is extendable therefrom and adapted to form an opening in the tubular prosthesis in the vicinity of the detected branch vessel.

30. The system of claim 29 wherein said imaging catheter includes a guidewire that is extendable from said piercing member.

31. The system of claim 29 wherein said tubular prosthesis has a tubular side wall and said side wall has an opening formed therein.

32. The system of claim 31 wherein said tubular prosthesis comprises a graft.

33. The system of claim 31 wherein said tubular prosthesis comprises a stent-graft.

34. The system of claim 29 wherein said tubular prosthesis comprises a graft.

35. The system of claim 29 wherein said tubular prosthesis comprises a stent-graft.

36. The system of claim 29 further including a tubular branch vessel prosthesis adapted for endovascular delivery to a branch vessel that branches from said vessel.
